(19) United States
(12) Patent Application Publication
(10) Pub. No.: US 2022/0257596 A1
(43) Pub. Date: Aug. 18, 2022

(54) METHODS FOR TREATING MEIBOMIAN GLAND DYSFUNCTION WITH LIVER X RECEPTOR AGONISTS

(71) Applicant: Novartis AG, Basel (CH)

(72) Inventors: Christopher M. ADAMS, Arlington, MA (US); Qian HUANG, Somerville, MA (US); Sandra TEIXEIRA, Charlestown, MA (US); Brett THIBODEAUX, Palm Bay, FL (US)

(21) Appl. No.: 17/625,201

(22) PCT Filed: Jul. 13, 2020
(86) PCT No.: PCT/IB2020/056584

§ 371 Date: Jan. 6, 2022

Related U.S. Application Data
(60) Provisional application No. 62/874,433, filed on Jul. 15, 2019.
(60) Provisional application No. 62/939,138, filed on Nov. 22, 2019.

(51) Int. Cl. A61K 31/506; A61K 31/405; A61K 31/454; A61K 31/195; A61K 31/497; A61K 31/18; A61K 31/415; A61K 31/4174; A61K 31/137; A61K 31/496; A61K 31/416; A61K 31/4545; A61K 9/00; A61P 27/14

(52) U.S. Cl. CPC A61K 31/506; A61K 31/405; A61K 31/454; A61K 31/195; A61K 31/497; A61K 31/18; A61P 27/14; A61K 31/4174; A61K 31/137; A61K 31/496; A61K 31/416; A61K 31/4545; A61K 9/0048; A61K 31/415

(57) ABSTRACT

The present invention provides methods for treating meibomian gland dysfunction using liver X receptor (LXR) agonists.

### FIELD OF THE INVENTION

The present disclosure relates to methods for treating meibomian gland dysfunction or ocular diseases or disorders using liver X receptor (LXR) agonists.

### BACKGROUND OF THE INVENTION

Tears are comprised of three layers. The mucus layer coats the cornea forming a foundation so the tear film can adhere to the eye. The middle aqueous layer provides moisture and supplies oxygen and other important nutrients to the cornea. The outer lipid layers is an oily film that seals the tear film on the eye and helps to prevent evaporation of the layers beneath.

Meibomian glands (located at the lid margins) are primarily responsible for lipid generation, and abnormal secretions from in these glands can lead to an unhealthy lipid layer in the tear film. The lipid secreted by the meibomian glands also retards evaporation from the preocular surface, lowers the surface tension of tears, prevents spillover of tears from the lid margin, prevents the contamination of the tear film by sebaceous lipids and prevents damage to the skin of the lid margin.

Dysfunction of the meibomian glands can lead to lipid insufficiency that destabilizes the tear film and causes decreases in tear film break-up time and evaporative dry eye (see, e.g., Sullivan et al., Ann. NY Acad. Sci., 966, 211-222, 2002). meibomian gland dysfunction may also be characterized by increased melting point of the lipids, causing solidification of the lipids and obstruction of the meibomian gland secretion. This can result in cysts, infections and decreased lipid content in the tears.

Commonly used treatments to treat meibomian gland dysfunction include warm compresses to eyelid margins, mechanical probing of meibomian ducts, using infrared devices or chemicals to eyelid margins to induce tear lipid melting and secretion. For inflammation, glucocorticoids may be used. If there is a bacterial component, antibiotics like penicillin, doxycycline and tetracyclines may be used. However, these therapies are not suitable for long term use. There is a long-felt and unmet need for safe, effective treatments for the treatment of meibomian gland dysfunction that can improve lipid quality and tear film.

The Liver X receptor (LXR) was first described by Willy, P. J., et al., “LXR, a nuclear receptor that defines a distinct retinoid response pathway,” Genes & Development 9:1033-1045 (Cold Spring Harbor Laboratory Press).

The liver X receptors (LXR alpha and LXR beta) are highly expressed in the epidermis. Activation of LXRs is known to improve permeability barrier homeostasis by a number of mechanisms, including stimulating epidermal lipid synthesis, increasing lamellar body formation and secretion, and increasing the activity of enzymes required for the extracellular processing of lipids in the stratum corneum, leading to the formation of lamellar membranes that mediate permeability barrier function.

Several LXR agonists are known in the literature and have been investigated for treatment of various disorders.

The present inventors found that use of agonists of the liver X receptor upregulated stearoyl-CoA desaturase-1 (SCD-1) in cutaneous cells and surprisingly decreased the melting point of secretions of the meibomian gland, thereby potentially relieving meibomian gland dysfunction.

### SUMMARY OF THE INVENTION

In some embodiments, the present invention provides a method of treating meibomian gland dysfunction (MGD) in a subject in need thereof, comprising administering an effective amount of a liver X receptor (LXR) agonist to the subject. In some embodiments, the LXR agonist is:
2-(4-(3-((2-chloro-3-(trifluoromethyl)benzyl)(2,2-diphenylethyl)amino)propoxy)-1H-indol-1-yl)acetic acid;
2-(tert-butyl)-5-phenyl-4-((4-(piperidin-1-yl)phenyl)amino)isothiazol-3(2H)-one 1,1-dioxide;
(R)-2-(3-(3-((2-chloro-3-(trifluoromethyl)benzyl)(2,2-diphenylethyl)amino)butoxy)phenyl)-2-methylpropanoic acid;
(R)-(2-(4-(4-(hydroxymethyl)-3-(methylsulfonyl)phenyl)-2-isopropylpiperazin-1-yl)-4-(trifluoromethyl)pyrimidin-5-yl)methanol;
2-(5-(methyl(3-((7-propyl-3-(trifluoromethyl)benzo[d]isoxazol-6-yl)oxy)propyl)amino)pyrazin-2-yl)acetic acid;
N-(4-(1,1,1,3,3,3-hexafluoro-2-hydroxypropan-2-yl)phenyl)-N-(2,2,2-trifluoroethyl)benzenesulfonamide;
ethyl 2-(5-(3′-(methylsulfonyl)-[1,1′-biphenyl]-4-yl)-3-(trifluoromethyl)-1H-pyrazol-1-yl)acetate;
2-(3-(3-((2-chloro-3-(trifluoromethyl)benzyl)(2,2-diphenylethyl)amino)propoxy)phenyl)acetic acid;
2-(2-(2-(2,6-dichlorophenyl)propan-2-yl)-1-(3,3′-difluoro-4′-(hydroxymethyl)-5′-(methylsulfonyl)-[1,1′-biphenyl]-4-yl)-1H-imidazol-4-yl)propan-2-ol;
2-(4-(benzyl(ethyl)amino)-3-chlorophenyl)-1,1,1,3,3,3-hexafluoropropan-2-ol;
2-chloro-4-(5-cyano-6-(4-(2-methyl-2-phenylpropanoyl)piperazin-1-yl)pyridin-3-yl)-N,N-dimethylbenzamide;
2-(2-chloro-4-fluorobenzyl)-3-(4-fluorophenyl)-7-(trifluoromethyl)-2H-indazole;
2-chloro-4-(1′-((2-chlorophenyl)sulfonyl)-[4,4′-bipiperidin]-1-yl)-N,N-dimethylbenzamide;
(R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol; or

salts, esters, or co-crystals thereof.

In particular embodiments, the LXR agonist is 2-(tert-butyl)-5-phenyl-4-((4-(piperidin-1-yl)phenyl)amino)isothiazol-3(2H)-one 1,1-dioxide; (R)-2-(3-(3-((2-chloro-3-(trifluoromethyl)benzyl)(2,2-diphenylethyl)amino)butoxy)phenyl)-2-methylpropanoic acid; ethyl 2-(5-(3′-(methylsulfonyl)-[1,1′-biphenyl]-4-yl)-3-(trifluoromethyl)-1H-pyrazol-1-yl)acetate; (R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol, or a salt, ester, or co-crystal thereof.

In some embodiments of the presently described methods, the invention comprises administering about 0.001 mg to about 50 mg of the LXR agonist to the subject. In some embodiments, the LXR agonist is ocularly administered to the subject. In particular embodiments, the ocular administration is to an eyelid, e.g., eyelid skin or the eyelid margin, of the subject. In further embodiments, the ocular administration is to the ocular surface, e.g., the cornea and/or the conjuctiva of the subject.

In some embodiments of the present invention, the administration of the LXR agonist results in an increase in the desaturation index of nonpolar lipids generated by human sebaceous gland cell line (SZ95) cells, when measured in vitro as described herein is increased by about 10% to about 200%, by about 10% to about 150%, by about 10% to about 100%. In particular embodiments, the desaturation index is increased by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, or about 200%.

In some embodiments, the administration of the LXR agonist results in a decrease in the melting temperature of meibum in the subject. In particular embodiments, the melting temperature of meibum is decreased by about 5, about 4, about 3, about 2, or about 1 degrees centigrade.

In some embodiments of the present invention, the subject is diagnosed with meibomian gland dysfunction or dry eye disease or ocular surface disease. In some embodiments, the administration decreases the signs and/or symptoms of meibomian gland dysfunction or dry eye disease or ocular surface disease. In particular embodiments, the administration of the LXR agonist results in one or more of the following (or similar or equivalent tests):

increased tear film break-up time of at least about 2, 3, 4, or 5 seconds;

meibomian gland expression grading improvement by 1 or 2 or 3 grades;

increased tear meniscus of at least about 10%;

decreased corneal fluorescein staining of at least about 10%, or

increased Schirmer test score of at least about 2 mm.

In particular embodiments, the administration results in reduced incidence of at least about 10% in one or more of the following signs and/or symptoms (or similar or equivalent signs and/or symptoms): ocular dryness, ocular discomfort or pain, eye itchiness, blurry vision, heavy or fatigued eyes, watery eyes, ocular hyperemia, ocular burning or stinging, grittiness or foreign body sensation, or photophobia or light sensitivity, crusty or red or swollen eyelids or eyelid margins, sensitivity to environmental factors such as wind or low humidity, or loss of tolerability to contact lens use.

In some embodiments, the methods of the present invention further comprise administering an additional therapeutic agent to the subject. In some embodiments, the additional therapeutic agent is a retinoid X receptor (RXR) agonist or an ophthalmic steroid. In particular embodiments, the RXR agonist is vitamin A, retinoic acid, phytanic acid, lithocholic acid, bexarotene, docosahexaenoic acid, or flurobexarotene. In some embodiments, the additional therapeutic agent is a retinoid X receptor (RXR) agonist or an ophthalmic steroid. In particular embodiments, the RXR agonist is vitamin A, retinoic acid, phytanic acid, lithocholic acid, bexarotene, docosahexaenoic acid, flurobexarotene, or pharmaceutically acceptable salts thereof. In particular embodiments, the ophthalmic steroid is dexamethasone, fluocinolone, loteprednol, difluprednate, fluorometholone, prednisolone, prednisone, medrysone, triamcinolone, betamethasone, rimexolone, or pharmaceutically acceptable salts thereof. Further non-limiting examples of such additional therapeutic agents include Xiidra® (lifitegrast), Restasis® (cyclosporine), minocycline, doxycycline, or other tetracycline antibiotics. Other examples include keratolytic agents such as selenium disulfide, salicylic acid, glycolic acid etc., or pharmaceutically acceptable salts thereof.

In some embodiments of the present invention, the invention is a method of upregulating stearoyl-CoA desaturase 1 (SCD1) in a subject suffering from meibomian gland dysfunction (MGD), comprising administering an liver X receptor (LXR) agonist to the subject. In some embodiments, the LXR agonist is:
2-(4-(3-((2-chloro-3-(trifluoromethyl)benzyl)(2,2-diphenylethyl)amino)propoxy)-1H-indol-1-yl)acetic acid;
2-(tert-butyl)-5-phenyl-4-((4-(piperidin-1-yl)phenyl)amino)isothiazol-3(2H)-one 1,1-dioxide;
(R)-2-(3-(3-((2-chloro-3-(trifluoromethyl)benzyl)(2,2-diphenylethyl)amino)butoxy)phenyl)-2-methylpropanoic acid;
(R)-(2-(4-(4-(hydroxymethyl)-3-(methylsulfonyl)phenyl)-2-isopropylpiperazin-1-yl)-4-(trifluoromethyl)pyrimidin-5-yl)methanol;
2-(5-(methyl(3-((7-propyl-3-(trifluoromethyl)benzo[d]isoxazol-6-yl)oxy)propyl)amino)pyrazin-2-yl)acetic acid;
N-(4-(1,1,1,3,3,3-hexafluoro-2-hydroxypropan-2-yl)phenyl)-N-(2,2,2-trifluoroethyl)benzenesulfonamide;
ethyl 2-(5-(3′-(methylsulfonyl)-[1,1′-biphenyl]-4-yl)-3-(trifluoromethyl)-1H-pyrazol-1-yl)acetate;
2-(3-(3-((2-chloro-3-(trifluoromethyl)benzyl)(2,2-diphenylethyl)amino)propoxy)phenyl)acetic acid;
2-(2-(2-(2,6-dichlorophenyl)propan-2-yl)-1-(3,3′-difluoro-4′-(hydroxymethyl)-5′-(methylsulfonyl)-[1,1′-biphenyl]-4-yl)-1H-imidazol-4-yl)propan-2-ol;
2-(4-(benzyl(ethyl)amino)-3-chlorophenyl)-1,1,1,3,3,3-hexafluoropropan-2-ol; 2-chloro-4-(5-cyano-6-(4-(2-methyl-2-phenylpropanoyl)piperazin-1-yl)pyridin-3-yl)-N,N-dimethylbenzamide;
2-(2-chloro-4-fluorobenzyl)-3-(4-fluorophenyl)-7-(trifluoromethyl)-2H-indazole;
2-chloro-4-(1′-((2-chlorophenyl)sulfonyl)-[4,4′-bipiperidin]-1-yl)-N,N-dimethylbenzamide;
(R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol;

salts, esters, or co-crystals thereof.

In particular embodiments, the LXR agonist is 2-(tert-butyl)-5-phenyl-4-((4-(piperidin-1-yl)phenyl)amino)isothiazol-3(2H)-one 1,1-dioxide; (R)-2-(3-(3-((2-chloro-3-(trifluoromethyl)benzyl)(2,2-diphenylethyl)amino)butoxy)phenyl)-2-methylpropanoic acid; ethyl 2-(5-(3′-(methylsulfonyl)-[1,1′-biphenyl]-4-yl)-3-(trifluoromethyl)-1H-pyrazol-1-yl)acetate; (R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol, or a salt, ester, or co-crystal thereof.

In some embodiments of the methods of the present invention, the method comprises administering about 0.001 mg to about 50 mg of the LXR agonist to the subject. In particular embodiments, the LXR agonist is ocularly administered to the subject. In yet particular embodiments, the ocular administration is to an eyelid, e.g., eyelid skin or the eyelid margin, of the subject. In further embodiments, the ocular administration is to the ocular surface, e.g, the cornea and/or the conjuctiva of the subject.

In some embodiments of the presently claimed methods, the LXR agonist is formulated in a pharmaceutically acceptable formulation.

In some embodiments of the present invention, the LXR agonist is formulated in a pharmaceutically acceptable formulation. In particular embodiments, the pharmaceutically acceptable formulation is a solution, suspension, gel, cream, ointment, liposomes, or ocular insert. In some embodiments, the concentration of the LXR agonist in the pharmaceutically acceptable formulation is about 0.01% w/w to about 10% w/w, or about 0.01% w/w to about 5% w/w, or about 0.05% to about 3% w/w, or about 0.05% w/w to about 0.5% w/w, or about 0.15% w/w, about 0.1% w/w, about 0.5% w/w, about 1.0% w/w about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, or about 6.0% w/w.

In some embodiments of the present invention, the administration of the LXR agonist results in an increase in the desaturation index of nonpolar lipids generated by human sebaceous gland cell line (SZ95) cells, when measured in vitro as described herein is increased by about 10% to about 200%, by about 10% to about 150%, by about 10% to about 100%. In particular embodiments, the desaturation index is increased by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, or about 200%.

In some embodiments, the administration results in a decrease in the melting temperature of meibum in the subject. In particular embodiments, the melting temperature of meibum is decreased by about 5, about 4, about 3, about 2, or about 1 degrees centigrade.

In some embodiments of the present invention, the subject is diagnosed with meibomian gland dysfunction or dry eye disease or ocular surface disease. In some embodiments, the administration decreases the signs and/or symptoms of meibomian gland dysfunction or dry eye disease or ocular surface disease. In particular embodiments, the administration results in one or more of the following (or similar or equivalent tests):

increased tear film break-up time of at least about 2, 3, 4, or 5 seconds;

meibomian gland expression grading improvement by 1 or 2 or 3 grades;

increased tear meniscus of at least about 10%;

decreased corneal fluorescein staining of at least about 10%, or

increased Schirmer test score of at least about 2 mm.

In particular embodiments, the administration results in reduced incidence of at least about 10% in one or more of the following signs and/or symptoms (or similar or equivalent signs and/or symptoms):ocular dryness, ocular discomfort or pain, eye itchiness, blurry vision, heavy or fatigued eyes, watery eyes, ocular hyperemia, ocular burning or stinging, grittiness or foreign body sensation, or photophobia or light sensitivity, crusty or red or swollen eyelids or eyelid margins, sensitivity to environmental factors such as wind or low humidity, loss of tolerability to contact lens use.

In some embodiments, the methods of the present invention further comprise administering an additional therapeutic agent to the subject. In some embodiments, the additional therapeutic agent is a retinoid X receptor (RXR) agonist or an ophthalmic steroid. In particular embodiments, the RXR agonist is vitamin A, retinoic acid, phytanic acid, lithocholic acid, bexarotene, docosahexaenoic acid, flurobexarotene, or pharmaceutically acceptable salts thereof. In particular embodiments, the ophthalmic steroid is dexamethasone, fluocinolone, loteprednol, difluprednate, fluorometholone, prednisolone, prednisone, medrysone, triamcinolone, betamethasone, rimexolone, or pharmaceutically acceptable salts thereof. Further non-limiting examples of such additional therapeutic agents include Xiidra® (lifitegrast), Restasis® (cyclosporine), minocycline, doxycycline, or other tetracycline antibiotics. Other examples include keratolytic agents such as selenium disulfide, salicylic acid, glycolic acid etc., or pharmaceutically acceptable salts thereof.

In some embodiments, the present invention comprises a method of reducing the symptoms of meibomian gland dysfunction (MGD) in a subject in need thereof, comprising administering an effective amount of a liver X receptor (LXR) agonist to the subject.

In some embodiments, the LXR agonist is:
2-(4-(3-((2-chloro-3-(trifluoromethyl)benzyl)(2,2-diphenylethyl)amino)propoxy)-1H-indol-1-yl)acetic acid;
2-(tert-butyl)-5-phenyl-4-((4-(piperidin-1-yl)phenyl)amino)isothiazol-3(2H)-one 1,1-dioxide;
(R)-2-(3-(3-((2-chloro-3-(trifluoromethyl)benzyl)(2,2-diphenylethyl)amino)butoxy)phenyl)-2-methylpropanoic acid;
(R)-(2-(4-(4-(hydroxymethyl)-3-(methylsulfonyl)phenyl)-2-(isopropylpiperazin-1-yl)-4-(trifluoromethyl)pyrimidin-5-yl)methanol;
2-(5-(methyl(3-((7-propyl-3-(trifluoromethyl)benzo[d]isoxazol-6-yl)oxy)propyl)amino)pyrazin-2-yl)acetic acid;
N-(4-(1,1,1,3,3,3-hexafluoro-2-hydroxypropan-2-yl)phenyl)-N-(2,2,2-trifluoroethyl)benzenesulfonamide;
ethyl 2-(5-(3′-(methylsulfonyl)-[1,1′-biphenyl]-4-yl)-3-(trifluoromethyl)-1H-pyrazol-1-yl)acetate;
2-(3-(3-((2-chloro-3-(trifluoromethyl)benzyl)(2,2-diphenylethyl)amino)propoxy)phenyl)acetic acid;
2-(2-(2-(2,6-dichlorophenyl)propan-2-yl)-1-(3,3′-difluoro-4′-(hydroxymethyl)-5′-(methylsulfonyl)-[1,1′-biphenyl]-4-yl)-1H-imidazol-4-yl)propan-2-ol;
2-(4-(benzyl(ethyl)amino)-3-chlorophenyl)-1,1,1,3,3,3-hexafluoropropan-2-ol; 2-chloro-4-(5-cyano-6-(4-(2-methyl-2-phenylpropanoyl)piperazin-1-yl)pyridin-3-yl)-N,N-dimethylbenzamide;
2-(2-chloro-4-fluorobenzyl)-3-(4-fluorophenyl)-7-(trifluoromethyl)-2H-indazole; 2-chloro-4-(1′-((2-chlorophenyl)sulfonyl)-[4,4′-bipiperidin]-1-yl)-N,N-dimethylbenzamide;
(R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol;

salts, esters, or co-crystals thereof.

In particular embodiments, the LXR agonist is 2-(tert-butyl)-5-phenyl-4-((4-(piperidin-1-yl)phenyl)amino)isothiazol-3(2H)-one 1,1-dioxide; (R)-2-(3-(3-((2-chloro-3-(trifluoromethyl)benzyl)(2,2-diphenylethyl)amino)butoxy)phenyl)-2-methylpropanoic acid; ethyl 2-(5-(3′-(methylsulfonyl)-[1,1′-biphenyl]-4-yl)-3-(trifluoromethyl)-1H-pyrazol-1-yl)acetate; (R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol, or a salt, ester, or co-crystal thereof.

In particular embodiments, the method comprises administering about 0.001 mg to about 50 mg of the LXR agonist to the subject.

In some embodiments, the LXR agonist is ocularly administered to the subject. In particular embodiments, the ocular administration is to an eyelid, e.g., eyelid skin or the eyelid margin, of the subject. In further particular embodiments, the ocular administration is to the ocular surface, e.g., the cornea and/or the conjuctiva of the subject.

### BRIEF DESCRIPTION OF THE DRAWINGS

FIG. 1 demonstrates the decrease in meibum melting temperature measured upon administration of an exemplary compound (Compound B) to rat eyes at a concentration of 1%, in comparison to vehicle.

FIG. 2 demonstrates the decrease in meibum melting temperature measured upon administration of an exemplary compound (Compound C) to rat eyes at a concentration of 1%, in comparison to vehicle.

### DETAILED DESCRIPTION

The language “effective amount” of the compounds described herein, refers to that amount of a therapeutic compound necessary or sufficient to perform its intended function within a mammal. An effective amount of the therapeutic compound can vary according to factors such as the amount of the causative agent already present in the mammal, the age, sex, and weight of the mammal, and the ability of the therapeutic compounds of the present disclosure to treat the ocular surface disorder and/or symptoms thereof in the mammal.

The phrase “ophthalmically compatible” refers to formulations, polymers and other materials and/or dosage forms which are suitable for use in contact with the ocular tissues of human beings and animals without excessive toxicity, irritation, allergic response, or other problem or complication, commensurate with a reasonable benefit/risk ratio.

As used herein, the term “treating” means to relieve, alleviate, delay, reduce, reverse, or improve at least one symptom of a condition in a subject. The term “treating” refers to relieving, alleviating, delaying, reducing, reversing, or improving at least one symptom selected from abnormal meibomian gland secretions, meibomian gland dysfunction, dry eye, meibomian gland secretions, redness of the eyelid mar gins, burning and/or itching in a subject's eye, ocular discomfort, corneal epithelial erosion, ocular and conjunctival staining, and reducing blurred and/or fuzzy vision. The term “treating” may also mean to arrest, delay the onset (i.e., the period prior to clinical manifestation of a disease) and/or reduce the risk of developing or worsening a condition.

As used herein, the term “subject” or “patient” refers to human and non-human mammals, including but, not limited to, primates, rabbits, pigs, horses, dogs, cats, sheep, and cows. In particular embodiments, a subject or patient is a human. In some embodiments, the term “patient” or “subject” refers to a human being who is diseased with the condition (i.e., disease or disorder) described herein and who would benefit from the treatment. As used herein, a subject is “in need of” a treatment if such subject (patient) would benefit biologically, medically or in quality of life from such treatment. In particular embodiments, the subject is an adult human at least about 18 years of age. In particular embodiments, the subject is an adult human from about 18 to about 75 years of age. In some embodiments, the subject is a human child up to about 18 years of age.

As used herein, “ocular surface” refers to the outer surface of the eye, which anatomically comprises the cornea (with epithelium, bowman layer, stroma, descement membrane, endothelium), conjunctiva, and the corneo-scleral junction, i.e. limbus.

As used herein, “Liver X receptor” or “LXR” refers to a nuclear receptor implicated in cholesterol biosynthesis. As used herein, the term LXR refers to both LXRa and LXRI3, two forms of the protein found in mammals, fragments or isoforms thereof.

As used herein, the term “stearoyl-CoA desaturase-1” or “SCD-1” refers to an enzyme that catalyzes a rate-limiting step in the synthesis of unsaturated fatty acids. As used herein, the term “desaturation index” refers to the ratio of desaturated fatty acids and esters compared to saturated fatty acids and esters for the SCD enzyme when measured using a sentinel lipid assay in vitro, for example, in SZ95 cells, as described herein.

As used herein, ocular hyperemia refers to redness of the ocular surface. Ocular hyperemia may be a clinical marker for inflammation and/or ocular irritation. Ocular hyperemia may be measured using the McMonnies scale, at values from 0 to 5, based on standard photographs.

As used herein, “meibomian gland expression grading” refers to a scale for assessing the severity of meibomian gland dysfunction, for example, as described in Tomlinson, Alan, et al.. (2011), “The International Workshop on meibomian Gland Dysfunction: Report of the Diagnosis Subcommittee,” Investigative Ophthalmology & Visual Science, vol. 52, no. 4, pp. 2006-2049.

As used herein, “placebo” refers to an ophthalmic formulation that includes all the components of the administered drug composition without the drug.

As used herein, the term “about” refers to a range of values+10% of a specified value.

As used herein, a pharmaceutical composition is a composition suitable for pharmaceutical use. A composition suitable for pharmaceutical use may be sterile, homogeneous and/or isotonic. Pharmaceutical compositions may be prepared in certain embodiments in an aqueous form, for example in a pre-filled syringe or other single- or multi-dose container. In certain embodiments of the invention, the pharmaceutical composition is ophthalmically compatible and suitable for ophthalmic administration to a human subject by, for example, topical or other known methods of delivery.

Any chemical formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the disclosure include, for example, isotopes of hydrogen, carbon, nitrogen, and oxygen, such as ³H, ¹¹C, ¹³C, ¹⁴C, and ¹⁵N. Accordingly, it should be understood that methods of the present invention can or may involve compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. Isotopically-labeled compounds can generally be prepared by conventional techniques known to those skilled in the art, e.g., using an appropriate isotopically-labeled reagents in place of the non-labeled reagent previously employed.

The present invention encompasses embodiments that include all pharmaceutically acceptable salts of the compounds useful according to the invention provided herein. As used herein, “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts of the parent compound formed, for example, from non-toxic inorganic or organic acids. The pharmaceutically acceptable salts can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety. For example, preferred pharmaceutically acceptable salts include, but are not limited to, mineral or organic acid salts of basic residues such as amines. For example, the salt can be a hydrochloride salt. Other examples of suitable salts can be found in U.S. Pat. No. 8,349,852, the content of which is hereby incorporated by its entirety.

The phrase “pharmaceutically acceptable” as employed herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present inventors discovered that in vivo administration of exemplary LXR agonists surprisingly decreased the melting point of secretions of the meibomian gland, thereby having the potential to treat or relieve or reduce symptoms of meibomian gland dysfunction and other ocular diseases or disorders (e.g., dry eye disease).

In some embodiments, the present invention includes methods of treating meibomian gland dysfunction, by administering an effective amount of an LXR agonist.

In some embodiments, the present invention includes methods of treating meibomian gland dysfunction, by administering an effective amount of one or more of the following compounds in the following Table:

| Compound | Structure | Name |
|---|---|---|
| Compound A | 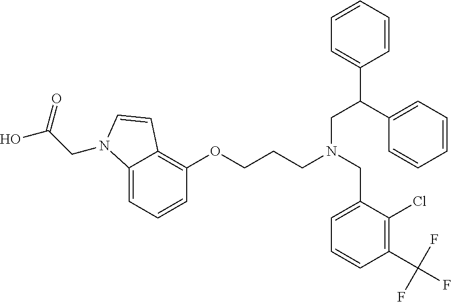 [SMILES: O=C(O)Cn1ccc2c(OCCCN(Cc3cccc(C(F)(F)F)c3Cl)CC(c3ccccc3)c3ccccc3)cccc21] | 2-(4-(3-((2-chloro-3- (trifluoromethyl)benzyl)(2,2- diphenylethyl)amino)propoxy)-1H-indol-1- yl)acetic acid |
| Compound B | 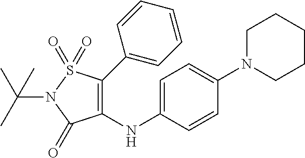 [SMILES: CC(C)(C)N1C(=O)C(Nc2ccc(N3CCCCC3)cc2)=C(c2ccccc2)S1(=O)=O] | 2-(tert-butyl)-5-phenyl-4-((4-(piperidin-1- yl)phenyl)amino)isothiazol-3(2H)-one 1,1- dioxide |
| Compound C | 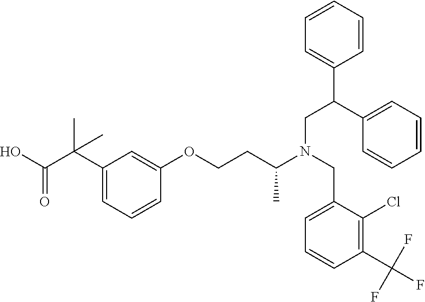 [SMILES: C[C@H](CCOc1cccc(C(C)(C)C(=O)O)c1)N(Cc1cccc(C(F)(F)F)c1Cl)CC(c1ccccc1)c1ccccc1] | (R)-2-(3-(3-((2-chloro-3- (trifluoromethyl)benzyl)(2,2- diphenylethyl)amino)butoxy)phenyl)-2- methylpropanoic acid |
| Compound D | 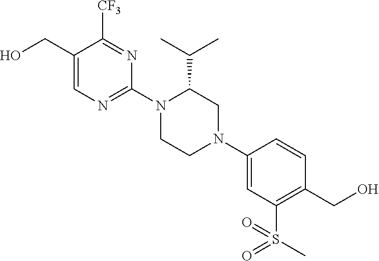 [SMILES: CC(C)[C@@H]1CN(c2ccc(CO)c(S(C)(=O)=O)c2)CCN1c1ncc(CO)c(C(F)(F)F)n1] | (R)-(2-(4-(4-(hydroxymethyl)-3- (methylsulfonyl)phenyl)-2- isopropylpiperazin-l-yl)-4- (trifluoromethyl)pyrimidin-5-yl)methanol |
| Compound E | 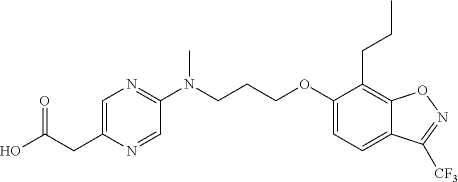 [SMILES: CCCc1c(OCCCN(C)c2cnc(CC(=O)O)cn2)ccc2c(C(F)(F)F)noc12] | 2-(5-(methyl(3-((7-propyl-3- (trifluoromethyl)benzo[d]isoxazol-6- yl)oxy)propyl)amino)pyrazin-2-yl)acetic acid |
| Compound F | 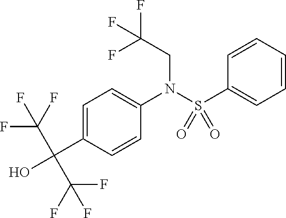 [SMILES: O=S(=O)(c1ccccc1)N(CC(F)(F)F)c1ccc(C(O)(C(F)(F)F)C(F)(F)F)cc1] | N-(4-(1,1,1,3,3,3-hexafluoro-2- hydroxypropan-2-yl)phenyl)-N-(2,2,2- trifluoroethyl)benzenesulfonamide |
| Compound G | 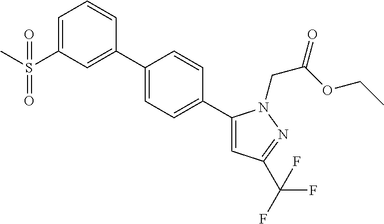 [SMILES: CCOC(=O)Cn1nc(C(F)(F)F)cc1-c1ccc(-c2cccc(S(C)(=O)=O)c2)cc1] | ethyl 2-(5-(3′-(methylsulfonyl)-[1,1′- biphenyl]-4-yl)-3-(trifluoromethyl)-1H- pyrazol-1-yl)acetate |
| Compound H | 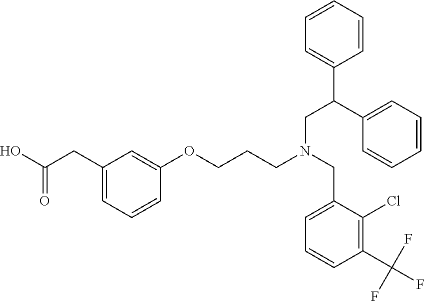 [SMILES: O=C(O)Cc1cccc(OCCCN(Cc2cccc(C(F)(F)F)c2Cl)CC(c2ccccc2)c2ccccc2)c1] | 2-(3-(3-((2-chloro-3- (trifluoromethyl)benzyl)(2,2- diphenylethyl)amino)propoxy)phenyl)acetic acid |
| Compound I | 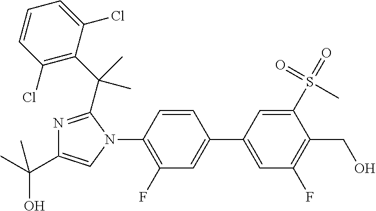 [SMILES: CC(C)(O)c1cn(-c2ccc(-c3cc(F)c(CO)c(S(C)(=O)=O)c3)cc2F)c(C(C)(C)c2c(Cl)cccc2Cl)n1] | 2-(2-(2-(2,6-dichlorophenyl)propan-2-yl)-1- (3,3′-difluoro-4′-(hydroxymethyl)-5′- (methylsulfonyl)-[1,1′-biphenyl]-4-yl)-1H- imidazol-4-yl)propan-2-ol |
| Compound J | 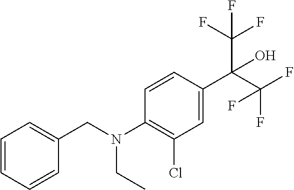 [SMILES: CCN(Cc1ccccc1)c1ccc(C(O)(C(F)(F)F)C(F)(F)F)cc1Cl] | 2-(4-(benzyl(ethyl)amino)-3-chlorophenyl)- 1,1,1,3,3,3-hexafluoropropan-2-ol |
| Compound K | 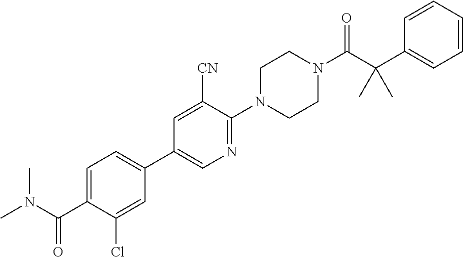 [SMILES: CN(C)C(=O)c1ccc(-c2cnc(N3CCN(C(=O)C(C)(C)c4ccccc4)CC3)c(C#N)c2)cc1Cl] | 2-chloro-4-(5-cyano-6-(4-(2-methyl-2- phenylpropanoyl)piperazin-1-yl)pyridin-3- yl)-N,N-dimethylbenzamide |
| Compound L | 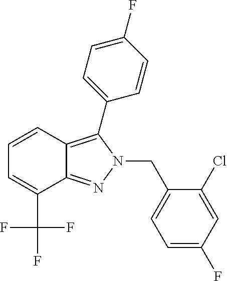 [SMILES: Fc1ccc(-c2c3cccc(C(F)(F)F)c3nn2Cc2ccc(F)cc2Cl)cc1] | 2-(2-chloro-4-fluorobenzyl)-3-(4- fluorophenyl)-7-(trifluoromethyl)-2H- indazole |
| Compound M | 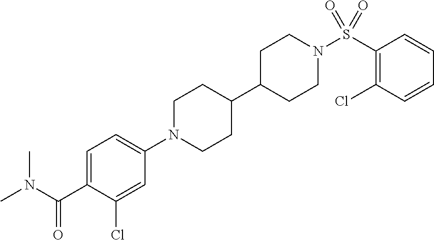 [SMILES: CN(C)C(=O)c1ccc(N2CCC(C3CCN(S(=O)(=O)c4ccccc4Cl)CC3)CC2)cc1Cl] | 2-chloro-4-(1′-((2-chlorophenyl)sulfonyl)- [4,4′-bipiperidin]-1-yl)-N,N- dimethylbenzamide |
| Compound N | 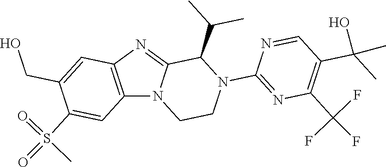 [SMILES: CC(C)[C@@H]1c2nc3cc(CO)c(S(C)(=O)=O)cc3n2CCN1c1ncc(C(C)(C)O)c(C(F)(F)F)n1] | (R)-2-(2-(8-(hydroxymethyl)-1-isopropyl- 7-(methylsulfonyl)-3,4- dihydrobenzo[4,5]imidazo[1,2-a]pyrazin- 2(1H)-yl)-4-(trifluoromethyl)pyrimidin-5- yl)propan-2-ol |

or salts, esters, or co-crystals thereof.

In some embodiments, the LXR agonist may be one or more compounds described in International Application Publication WO2005/023196, incorporated by reference herein.

In some embodiments, the LXR agonist may be one or more compounds described in International Application Publication WO2006/073363, incorporated by reference herein.

In some embodiments, the LXR agonist may be one or more compounds described in International Application Publication WO2003/082802, incorporated by reference herein.

In some embodiments, the LXR agonist may be one or more compounds described in International Application Publication WO2016/022521, incorporated by reference herein.

In some embodiments, the LXR agonist may be one or more compounds described in US Application Publication US2006/0178398, incorporated by reference herein.

In some embodiments, the LXR agonist may be one or more compounds described in International Application Publication WO2000/054759, incorporated by reference herein.

In some embodiments, the LXR agonist may be one or more compounds described in International Application Publication WO2013/130892, incorporated by reference herein.

In some embodiments, the LXR agonist may be one or more compounds described in International Application Publication WO2002/024632, incorporated by reference herein.

In some embodiments, the LXR agonist may be one or more compounds described in International Application Publication WO2010/138598, incorporated by reference herein.

In some embodiments, the LXR agonist may be one or more compounds described in International Application Publication WO2006/000323, incorporated by reference herein. In some embodiments, the LXR agonist may be one or more compounds described in International Application Publication WO2017/083216, incorporated by reference herein.

In some embodiments, the LXR agonist may be one or more compounds described in US Application Publication US2006/030612, incorporated by reference herein.

In some embodiments, the LXR agonist may be one or more compounds described in International Application Publication WO2017/083219, incorporated by reference herein.

In some embodiments, the LXR agonist may be one or more compounds described in International Application Publication WO2013/138568, incorporated by reference herein.

In some embodiments, about 0.001 mg to about 50 mg of the LXR agonist may be administered to the subject. In particular embodiments, a total dose of about 0.001 to about to about 50 mg of the LXR agonist may be administered to the subject per day. In some embodiments, the LXR agonist may be administered to the subject in one, two, three, four, or five divided doses per day. In particular embodiments, the LXR agonist may be administered to the subject once every one, two, three, four, five, six, or seven days. In some embodiments, the LXR agonist may be administered for at least a week, four weeks, or more. In particular embodiments, the LXR agonist may be administered for up to about 12 weeks, or greater than about 12 weeks.

In some embodiments, the LXR agonist is administered to the eye of the subject. Administration to the eye includes administration to all parts of the eye including all parts of the ocular surface such as the cornea, conjunctiva, and the corneo-scleral junction, i.e. limbus. In some embodiments, the LXR agonist is administered to the eyelid of the subject. Administration to the eyelid includes administration individually to the upper or lower eyelids, the eyelid skin or the eyelid margin, or both.

In some embodiments, the subject to be treated suffers from meibomian gland dysfunction. The meibomian gland is a holocrine type of exocrine gland, at the rim of the eyelid inside the tarsal plate, responsible for the supply of meibum, an oily substance that prevents evaporation of the eye's tear film. Meibomian gland dysfunction (MGD), also known as meibomitis, posterior blepharitis or inflammation of the meibomian glands, is a chronic, diffuse abnormality of the meibomian glands, commonly characterized by terminal duct obstruction and/or qualitative/quantitative changes in the glandular secretion (Nelson J D, et al., Invest Ophthalmol Vis Sci 2011; 52:1930-7). It may result in alteration of the tear film, symptoms of eye irritation, clinically apparent inflammation, and ocular surface disease. MGD often causes dry eye, and may contribute to blepharitis. In some cases topical steroids and topical/oral antibiotics are also prescribed reduce inflammation. Intense pulsed light (IPL) treatments or other mechanical treatments that apply heat and pressure to express the glands (eg, LipiFlow) have also been shown to reduce inflammation and improve the gland function in patients.

In some embodiments, the subject to be treated suffers from blepharitis. Blepharitis is an inflammatory condition of the eyelid margin, which can lead to permanent alterations in the eyelid margin or vision loss from superficial keratopathy, corneal neovascularization, and ulceration. According to anatomic location, blepharitis can be divided into anterior and posterior. Anterior blepharitis affects the eyelid skin, base of the eyelashes, and the eyelash follicles and includes the traditional classifications of staphylococcal and seborrheic blepharitis. Posterior blepharitis affects the meibomian glands and gland orifices, the primary cause being meibomian gland dysfunction. Symptoms of chronic blepharitis may include redness, burning sensation, irritation, tearing, eyelid crusting and sticking, and visual problems such as photophobia and blurred vision. Long-term management of symptoms may include daily eyelid cleansing routines and the use of therapeutic agents that reduce infection and inflammation. Treatment includes topical or systemic antibiotics e.g., bacitracin or erythromycin; oral antibiotics, e.g., tetracyclines (tetracycline, doxycycline, minocycline) or macrolides (erythromycin, azithromycin); topical steroids, e.g., corticosteroid, e.g., loteprednol etabonate, fluorometholone; topical combinations of an antibiotic and corticosteroid such as tobramycin/dexamethasone or tobramycin/loteprednol; topical cyclosporine 0.05%.

In some embodiments, the symptoms of a patient are assessed by asking the patient a series of questions. Questionnaires allow the assessment of a range of symptoms associated with ocular discomfort. In some embodiments, the questionnaire is the SPEED questionnaire. The SPEED questionnaire assesses frequency and severity of a patient's dry eye symptoms. It examines the occurrence of symptoms on the current day, past 72 hours and past three months. A SPEED score is tallied based on the patient's answers to the questions, to give a range of severity of the patient's symptoms. The SPEED questionnaire includes questions such as the following: 1) what dry eye symptoms are you experiencing, and when do they occur? 2) how frequently do you experience dryness, grittiness, or scratchiness in your eyes? 3) how often do you experience soreness or irritation of the eyes? 4) how often do you experience burning or watering of the eyes? 5) how often do you experience eye fatigue? and 6) how severe are the symptoms? In some embodiments, the questionnaire is the IDEEL questionnaire, which is similar to the SPEED questionnaire described herein.

Meibomian gland expressibility is optionally determined to assess the meibomian gland function. In normal patients, meibum is a clear to light yellow oil. Meibum is excreted from the glands when digital pressure is placed on the glands. Changes in meibomian gland expressibility are one potential indicator of MGD. In some embodiments, during expression, quantifying the amount of physical force applied during expression is monitored in addition to assessing lipid volume and lipid quantity.

Tear stability break up time (TBUT) is a surrogate marker for tear stability. Tear film instability is a core mechanism in dry eye and MGD. Low TBUT implies a possibility of lipid layer compromise and MGD. TBUT is optionally measured by examining fluorescein breakup time, as defined as the time to initial breakup of the tear film after a blink. Fluorescein is optionally applied by wetting a commercially available fluorescein-impregnated strip with saline, and applied to the inferior fornix or bulbar conjuctiva. The patient is then asked to blink several times and move the eyes. The break up is then analyzed with a slit lamp, a cobalt blue filter, and a beam width of 4 mm. The patient is instructed to blink, and the time from upstroke of the last blink to the first tear film break or dry spot formation is recorded as a measurement.

Other methods for assessing MGD signs and/or symptoms, include but are not limited to: Schirmer test, ocular surface staining, lid morphology analysis, meibography, meibometry, interferometry, evaporimetry, tear lipid composition analysis, fluorophotometry, meiscometry, lipid layer thickness, meibum desaturation index, meibomian gland loss osmolarity analysis, indices of tear film dynamics, reading speed, evaporation and tear turnover. Analysis of MGD signs and/or symptoms is performed by commonly understood methods known to those of skill in the art.

In certain embodiments, meibomian gland dysfunction is associated with one or more ocular diseases or disorders such as dry eye disease, ocular surface disease, Sjogren's Syndrome, conjunctivitis (including keratoconjuctivitis, vernal keratoconjunctivitis, allergic conjunctivitis), acanthamoeba, fibromyalgia, thyroid eye disease, rosacea, ptosis, keratoconus, ocular pain syndrome, Steven-Johnson's syndrome, corneal epitheliopathies, corneal neuropathies (including LASIK induced corneal neuropathies), corneal dystrophies (including recurrent corneal dystrophies), Map-Dot-Fingerprint Dystrophy, epithelial basement membrane dystrophy, corneal erosions or abrasions (including recurrent corneal erosions or abrasions), blepharitis (anterior, posterior, Demodex mites), graft vs host disease, meibomitis, glaucoma, conjunctivochalasis, keratopathies (including herpetic keratopathy, filamentary keratopathy, band or bullous keratopathy, exposure keratopathy), keratitis (including herpes simplex virus keratitis), iritis, episclentis, corneal surgery, multiple sclerosis, trichiasis, pterygium, Chordelum (internal or external), stye, neuralgia, or patients recovering from neurotrophic keratitis.

In some embodiments of the presently described methods, the administration of the LXR agonist reduces the signs and/or symptoms of meibomian gland dysfunction. Thus, in some embodiments, the invention results in a decrease of at least about 10%, at least about 15%, at least about 20%, or at least about 30% in the symptoms of dry eye disease, including one or more of the following signs and/or symptoms (or similar or equivalent signs and/or symptoms): ocular dryness, ocular discomfort or pain, eye itchiness, blurry vision, heavy or fatigued eyes, watery eyes, ocular hyperemia, ocular burning or stinging, grittiness or foreign body sensation, or photophobia or light sensitivity, crusty or red or swollen eyelids or eyelid margins, sensitivity to environmental factors such as wind or low humidity, loss of tolerability to contact lens use.

In some embodiments of the methods described herein, the administration of the LXR agonist does not result in a change (e.g., of less than 5% difference, less than 4% difference, or less than 3% difference) in one or more of best corrected visual acuity, slit-lamp biomicroscopy, dilated eye exam, intraocular pressure, compared to a placebo.

Pharmaceutical Compositions

The LXR agonists described herein may be administered alone or as an active ingredient of a formulation. Thus, the present invention also includes administration of pharmaceutical compositions or pharmaceutical product of compounds described herein, containing, for example, one or more pharmaceutically acceptable carriers. Methods of preparing various formulations are known to those of skill in the art and may be described in, for example, in the Handbook of Pharmaceutical Excipients, American Pharmaceutical Association (current edition); Pharmaceutical Dosage Forms Tablets (Lieberman, Lachman and Schwartz, editors) current edition, published by Marcel Dekker, Inc., as well as Remington's Pharmaceutical Sciences (Arthur Osol, editor), 1553-1593 (current edition).

The mode of administration and dosage form is closely related to the therapeutic amounts of the compounds or compositions which are desirable and efficacious for the given treatment application. Dosage forms include, but are not limited to, oral, rectal, sub-lingual, mucosal, nasal, ophthalmic, subcutaneous, intramuscular, topical, intravenous, transdermal, spinal, intrathecal, intra-articular, intra-arterial, subarachinoid, bronchial, lymphatic, and intra-uterine administration, and other dosage forms for systemic delivery of active ingredients. In particular embodiments, the dosage form is suitable for ocular administration. To prepare pharmaceutical dosage forms, the active ingredient may be mixed with a pharmaceutical carrier according to conventional pharmaceutical compounding techniques. The carrier may take a wide variety of forms depending on the form of preparation desired for administration.

In some embodiments, the pharmaceutical compositions are formulated as solutions, suspensions, gel, cream, ointment, liposomes, or ocular insert or other dosage forms suitable, in certain embodiments, for topical administration to the ocular surface, the cornea, the eyelid, margins of the eye, eye lashes and/or eye lid margin in order to deliver the formulation to the meibomian gland. In certain embodiments, liquid (aqueous or non-aqeuous) solutions may be used. Application of the formulation may be performed with an applicator, such as the patient's finger, a Weck-Cel®, Q-tip, or other device capable of delivering a formulation to the eyelid, eye lashes and/or eyelid margin in order to deliver the formulation to the meibomian gland. The formulations may be viscous or semi-viscous; liquid, solid, or semi-solid; aqueous or non-aqueous, depending on the site of application, dose, solubility of drug, and a variety of other factors that are considered by those of skill in the art.

Any of a variety of carriers may be used in a formulation used in the present invention. In one embodiment, the carrier is a non-aqueous carrier (e.g., oil, or oil mixture) having a viscosity in a range from about 50 cps to about 1000 cps, about 50 cps to about 500 cps, about 50 cps to about 200 cps, or about 60 cps to about 120 cps. In certain embodiments, the non-aqueous carrier comprises an oil, e.g., vegetable oils, silicone oils, mineral oil or any combination thereof. In some embodiments, the carrier may be liquid paraffin, white petrolatum, purified lanolin, gelation hydrocarbon, polyethylene glycol, hydrophilic ointment base, white ointment base, absorptive ointment base, Macrogol ointment base, simple ointment base, and the like. In certain embodiments, the formulation may include a monomeric polyol such as, glycerol, propylene glycol, and ethylene glycol, polymeric polyols such as polyethylene glycol, cellulose esters such hydroxypropylmethyl cellulose, carboxy methylcellulose sodiumand hydroxy propylcellulose; dextrans such as dextran 70; water soluble proteins such as gelatin, polymers such as polyvinyl alcohol, polyvinylpyrrolidone, and povidone; carbomers, such as carbomer 934P. carbomer 941, carbomer 940 and carbomer 974P; and gums such as HP-guar.

Additional excipients may optionally be included in the formulations of the present invention. Examples of additional excipients include, for example, tonicity enhancers, preservatives, solubilizers, non-toxic excipients, demulcents, sequestering agents, pH adjusting agents, co-solvents, viscosity building agents, and combinations thereof.

For the adjustment of the pH, for example to a physiological pH, buffers may be used. In certain embodiments, the pH of the formulation is maintained within the range of about 4.0 to about 8.0, such as, about 4.0 to about 6.0, for example, about 6.5 to about 7.8. Suitable buffers may be added, such as, e.g., boric acid, Sodium borate, potassium citrate, citric acid, sodium bicarbonate, TRIS, and various mixed phosphate buffers (including combinations of NaHPO, NaH2PO and KHPO) and mixtures thereof. Generally, buffers may be used in amounts ranging from about 0.05 to about 2.5 percent by weight, such as, from about 0.1 to about 1.5 percent by weight.

Tonicity may be adjusted, if needed, by the use of tonicity enhancing agents. Such agents may, for example, be of ionic and/or non-ionic type. Examples of ionic tonicity enhancers include, for example, alkali metal or earth metalhalides. Such as, for example, CaCl, KBr. KCl, LiCl, NaI, NaBr or NaCl, Na₂SO₄ or boric acid. Non-ionic tonicity enhancing agents include, for example, to urea, glycerol, sorbitol, mannitol, propylene glycol, or dextrose. In one embodiment, the formulations may have an osmolality of about 225 to about 400 mOsm/kg. In one embodiment, an osmolality of about 280 to about 320 mOsm is obtained.

In further embodiments, topical formulations may additionally comprise a preservative. A preservative may typically be selected from a quaternary ammonium compound such as benzalkonium chloride, benzoxonium chloride (e.g., N-benzyl-N—(C₈-C₁₈ dimethylammonium chloride) or the like.

Examples of preservatives different from quaternary ammonium salts include, for example, alkyl-mercury salts of thiosalicylic acid, such as, for example, thiomersal, phenylmercuric nitrate, phenylmercuric acetate or phenylmercuric borate, sodium perborate, sodium chlorite, parabens, such as, for example, methylparaben or propylparaben, alcohols, such as, for example, chlorobutanol, benzyl alcohol or phenylethanol, guanidine derivatives, such as, for example, chlorohexidine or polyhexamethylene biguanide, sodium perborate, or sorbic acid.

Where appropriate, a sufficient amount of preservative may be added to the ophthalmic composition to ensure protection against secondary-contaminations during use caused by bacteria and fungi.

In another embodiment, the formulations do not include a preservative.

The formulations described herein may additionally include a solubilizer. Suitable solubilizers include, but are not limited to, tyloxapol, fatty acid glycerol polyethylene glycol esters, fatty acid polyethylene glycol esters, polyethylene glycols, glycerol ethers, or cyclodextrins.

The formulations may further comprise non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. The amount and type of excipient added is in accordance with the particular requirements and is generally in the range of from approximately 0.0001 to approximately 90% by weight. Other compounds may also be added to the formulations of the present invention to adjust (e.g., increase) the viscosity of the carrier. Examples of viscosity enhancing agents include, but are not limited to, polysaccharides, such as hyaluronic acid and its salts, chondroitin sulfate and its salts, dextrans, various polymers of the cellulose family; vinyl polymers; and acrylic acid polymers.

In some embodiments, the formulations include an LXR agonist. In some embodiments of the present invention, the LXR agonist is at least one of:
2-(4-(3-((2-chloro-3-(trifluoromethyl)benzyl)(2,2-diphenylethyl)amino)propoxy)-1H-indol-1-yl)acetic acid (compound A);
2-(tert-butyl)-5-phenyl-4-((4-(piperidin-1-yl)phenyl)amino)isothiazol-3(2H)-one 1,1-dioxide (compound B);
(R)-2-(3-(3-((2-chloro-3-(trifluoromethyl)benzyl)(2,2-diphenylethyl)amino)butoxy)phenyl)-2-methylpropanoic acid (compound C);
(R)-(2-(4-(4-(hydroxymethyl)-3-(methylsulfonyl)phenyl)-2-isopropylpiperazin-1-yl)-4-(trifluoromethyl)pyrimidin-5-yl)methanol (compound D);
2-(5-(methyl(3-((7-propyl-3-(trifluoromethyl)benzo[d]isoxazol-6-yl)oxy)propyl)amino)pyrazin-2-yl)acetic acid (compound E);
N-(4-(1,1,1,3,3,3-hexafluoro-2-hydroxypropan-2-yl)phenyl)-N-(2,2,2-trifluoroethyl)benzenesulfonamide (compound F);
ethyl 2-(5-(3′-(methylsulfonyl)-[1,1′-biphenyl]-4-yl)-3-(trifluoromethyl)-1H-pyrazol-1-yl)acetate (compound G);
2-(3-(3-((2-chloro-3-(trifluoromethyl)benzyl)(2,2-diphenylethyl)amino)propoxy)phenyl)acetic acid (compound H);
2-(2-(2-(2,6-dichlorophenyl)propan-2-yl)-1-(3,3′-difluoro-4′-(hydroxymethyl)-5′-(methylsulfonyl)-[1,1′-biphenyl]-4-yl)-1H-imidazol-4-yl)propan-2-ol (compound I);
2-(4-(benzyl(ethyl)amino)-3-chlorophenyl)-1,1,1,3,3,3-hexafluoropropan-2-ol (compound J);
2-chloro-4-(5-cyano-6-(4-(2-methyl-2-phenylpropanoyl)piperazin-1-yl)pyridin-3-yl)-N,N-dimethylbenzamide (compound K);
2-(2-chloro-4-fluorobenzyl)-3-(4-fluorophenyl)-7-(trifluoromethyl)-2H-indazole (compound L);
2-chloro-4-(1′-((2-chlorophenyl)sulfonyl)-[4,4′-bipiperidin]-1-yl)-N,N-dimethylbenzamide (compound M);
(R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol (compound N),

or salts, esters, or co-crystals thereof.

In some embodiments, the LXR agonist is present in the formulation at a concentration of about 0.01% w/w to about 10% w/w, or about 0.01% w/w to about 5% w/w, or about 0.05% to about 3% w/w, or about 0.05% w/w to about 0.5% w/w, or about 0.15% w/w, about 0.1% w/w, about 0.5% w/w, about 1.0% w/w about 1.5% w/w or about 2.0% w/w.

In particular embodiments, the formulation includes 2-(tert-butyl)-5-phenyl-4-((4-(piperidin-1-yl)phenyl)amino)isothiazol-3(2H)-one 1,1-dioxide; (R)-2-(3-(3-((2-chloro-3-(trifluoromethyl)benzyl)(2,2-diphenylethyl)amino)butoxy)phenyl)-2-methylpropanoic acid; ethyl 2-(5-(3′-(methylsulfonyl)-[1,1′-biphenyl]-4-yl)-3-(trifluoromethyl)-1H-pyrazol-1-yl)acetate; (R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol, or a salt, ester, or co-crystal thereof, at a concentration of about 0.05% w/w, about 0.15% w/w, or 1.0% w/w.

In particular embodiments, the formulation including an LXR agonist is a gel, ointment, or a thermogelling formulation.

The LXR agonists will normally be contained in these formulations in an amount from about 0.1% to about 10.0% w/w. In some embodiments, concentrations of the LXR agonists for administration range from about 0.5% to about 1.5% w/w, about 0.5% to about 2.5% w/w, about 0.5% to about 3.5% w/w, about 0.5% to about 3.0% w/w, about 1.0% to about 2.5% w/w, about 1.5% to about 6.0% w/w, about 0.5% to about 5.0 w/w. In some embodiments, the concentration of the LXR agonists in a formulation for topical use is at least about 0.5% w/w, at least about 1.0% w/w, at least about 1.5% w/w, at least about 2.0% w/w, at least about 2.5% w/w, about 3.0% w/w, about 3.5% w/w, about 4.0% w/w, about 4.5% w/w, about 5.0% w/w, about 5.5% w/w, or about 6.0% w/w. In some embodiments, the concentration of the LXR agonists in a formulation for topical use is no more than about 6.0% w/w, no more than about 4.5% w/w, no more than about 4.0% w/w, no more than about 3.5% w/w, or no more than about 3.0% w/w. In particular embodiments, the concentration of LXR agonists in a formulation for topical use is about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, or about 6.0% w/w.

In some embodiments, the formulations are delivered to the surface of the eye one to six times a day, depending on the routine discretion of the skilled clinician. In some embodiments, the formulations are administered, one, two, three, or four times a day.

Unless otherwise specified, the weight or dosage referred to herein for the LXR agonists is the weight or dosage of the compound itself, not that of a salt or prodrug thereof, which can be different to achieve the intended therapeutic effect. For example, the weight or dosage of a corresponding salt of a compound suitable for the methods, compositions, or combinations disclosed herein may be calculated based on the ratio of the molecular weights of the salt and compound itself.

LXR agonists and/or pharmaceutically acceptable salts thereof may be incorporated into ophthalmically compatible formulations for delivery to the eye. The compounds may be combined with ophthalmologically acceptable preservatives, surfactants, viscosity enhancers, penetration enhancers, buffers, sodium chloride, and water to form an aqueous, sterile ophthalmic suspension or solution.

The pharmaceutical formulations may include an additional therapeutic agent in addition to LXR agonists. Further therapeutic agents may include, for instance, other compounds and antibodies useful for treating ocular disorders. A non-limiting list of such agents incudes retinoid X receptor agonists, such as vitamin A, retinoic acid, phytanic acid, lithocholic acid, bexarotene, docosahexaenoic acid, or flurobexarotene. In some embodiments, the additional therapeutic agent is a retinoid X receptor (RXR) agonist or an ophthalmic steroid. In particular embodiments, the RXR agonist is vitamin A, retinoic acid, phytanic acid, lithocholic acid, bexarotene, docosahexaenoic acid, flurobexarotene, or pharmaceutically acceptable salts thereof. In particular embodiments, the ophthalmic steroid is dexamethasone, fluocinolone, loteprednol, difluprednate, fluorometholone, prednisolone, prednisone, medrysone, triamcinolone, betamethasone, rimexolone, or pharmaceutically acceptable salts thereof. Further non-limiting examples of such additional therapeutic agents that may be included in the pharmaceutical composition include Xiidra® (lifitegrast), Restasis® (cyclosporine), minocycline, doxycycline, or other tetracycline antibiotics. Other examples include keratolytic agents such as selenium disulfide, salicylic acid, glycolic acid etc., or pharmaceutically acceptable salts thereof.

In some embodiments, the invention provides for the administration of LXR agonists to a subject in need thereof in a ophthalmically compatible formulation at a concentration of about 0.5% w/w to about 6.0% w/w. In some embodiments, concentrations for administration range from about 0.5% to about 3.5% w/w, about 0.5% to about 2.5% w/w, about 0.5% to about 1.5% w/w, about 0.5% to about 3.0% w/w, about 1.0% to about 2.5% w/w, about 1.5% to about 3.0% w/w, about 0.5% to about 2.5% w/w. In particular embodiments, the concentration of the LXR agonist in a formulation for topical use is about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, or about 6.0% w/w. In some embodiments, the LXR agonist is administered to the subject one to six times a day, e.g., one, two, three, or four times a day.

### EXAMPLES

The following examples are included to demonstrate nonlimiting embodiments of the present invention.

### Example 1. Measurement of SCD1 Expression in SZ95-SCD1-HiBit Cells

SZ95-SCD1-HiBit cells were seeded in 384-well cell culture white plates at a density of 3000 cells/30 ul. Water is added to edge wells to avoid evaporation. Cells were incubated in a humidified incubator with 5% CO₂ at 37° C. overnight. Tested compounds were diluted at a ratio of 1:3 using Agilent BRAVO Automated Liquid Handling Platform and added to cells at final concentrations starting from 18 μM. Compound H was used as a reference compound in each plate. Cells in the assay plate were incubated in a humidified incubator with 5% CO₂ at 37° C. for 48h.

The assay plates were removed from the incubator and allowed to equilibrate to room temperature. Nano-Glo® HiBiT Detection Reagent (Promega; a mixture of Nano-Glo HiBiT Detection Buffer, Nano-Glo HiBiT Detection Substrate, and LgBiT protein) was added into assay plates, at a volume equal to cell culture medium in each well. Plates were placed on an orbital shaker at a speed of 300-600 rpm for 10 min at room temperature, and read on an EnVision Plate Reader using luminescence detection with a 1 second read time.

The assay measures the increase in SCD1 protein production in vitro. Results are shown in Table 1 below. Aₘₐₓ refers to the percent EC₅₀ of the tested compound compared to the reference compound.

TABLE 1
Results from HiBiT assay

| Compound # | HiBit EC₅₀ (uM) | HiBit Aₘₐₓ |
|---|---|---|
| Compound F | 0.0474 | 76.1 |
| Compound H | 0.1131 | 92.0 |
| Compound L | >18.0 | 48.4 |
| Compound D | 0.0020 | 66.2 |
| Compound K | 0.5381 | 66.0 |
| Compound M | >18.0 | 39.1 |
| Compound B | 0.0018 | 64.0 |
| Compound G | 0.1106 | 70.7 |
| Compound I | >18.0 | 43.3 |
| Compound A | 0.0049 | 94.0 |
| Compound J | 0.3229 | 77.5 |
| Compound C | 0.0009 | 69.9 |
| Compound E | 0.0294 | 77.9 |
| Compound N | 0.004 | 72.4 |

### Example 2. Sentinel Lipid Assay

SZ95 (immortalized human sebaceous gland cells) cells were seeded in Greiner bio-one 96-well polypropylene plates that were pre-treated with 50 μg/ml Human Plasma Fibronectin (Thermo Fisher Scientific) at a density of 10⁴ cells/135 μl. Cells were incubated in a humidified incubator with 5% CO₂ at 37° C. overnight. Test compounds were diluted at a ratio of 1:3 and added to cells at final concentrations starting from 10 μM. Compound H was used as a positive control reference compound in each plate. Cells in the assay plate were incubated in a humidified incubator with 5% CO₂ at 37° C. for 72h.

Culture medium was removed from the cells and cells in culture plates were washed with ice cold phosphate buffered saline three times. Plates were heat sealed and stored in a −80° C. freezer prior to Sentinel lipid assay.

Sentinel lipid assay: The sentinel lipid assay was used quantify the change in the global desaturation index in SZ95 sebocytes upon administration of LXR agonist compounds. The assay measures a smaller subset of lipid analytes in meibum (termed “sentinel lipids”) which would model the global changes the population of both saturated and desaturated lipids in the cells. In order to define this smaller subset of lipids, a complete lipid profile was recorded on dose response curves (eight levels from 4 nM to 10 uM) of Compounds A-N. An elastic net regression model was applied separately to both the saturated and desaturated lipids to determine the minimum combination of coefficients and analytes which could be used to adequately model the total population of lipids. The elastic net model was able to reduce the behavior of 425 lipids to 11 lipids and the correlation between the desaturation indices observed using the complete set of lipids with from the 11 sentinel lipids was 0.96.

A medium throughput assay was created using this reduced set of sentinel lipids. A single batch is defined as triplicate examples of three unique plates (i.e., a single batch of cells is used to create nine plates for LC-MSMS analysis). Lipids were extracted from the cells using a 1:1 mixture of methylene chloride/methanol containing 10 nM of deuterated standards of triglycerides, which are used as internal standards for quantitating the lipid abundance. The lipids were separated prior to mass spectrometric analysis using a five minute HPLC gradient. The abundance of the sentinel lipids and the internal standards are measured using multiple reaction monitor mode (MRM) on a triple quadrupole mass spectrometer. The data was transformed from total ion current to nmoles/10⁶ cells, which are multiplied by the coefficients from the elastic net model to determine the effective desaturated and saturated content, and therein the desaturation index of the dosed cells. In order to compare compounds from multiple batches with one another, the measure raw desaturation index was normalized by dividing it by the desaturation index of the DMSO dosed cells, and all data was assessed as the fraction by which the compound increases the desaturation index above 1. Results from the sentinel lipid assay are shown in Table 2. The Aₘₐₓ value refers to the percent EC₅₀ of the tested compound compared to the reference compound.

TABLE 2
Results from the sentinel lipid assay

| Compound # | SLA EC₅₀ (uM) | SLA Aₘₐₓ |
|---|---|---|
| Compound F | 0.1901 | 89.8 |
| Compound H | 0.0881 | 97.4 |
| Compound L | >10.0 | 33.3 |
| Compound D | 0.0418 | 65.6 |
| Compound K | 0.4579 | 151.3 |
| Compound M | >10.0 | 29.2 |
| Compound B | 0.1679 | 92.0 |
| Compound G | 0.2428 | 98.8 |
| Compound I | 4.5196 | 60.2 |
| Compound A | 0.0073 | 104.4 |
| Compound J | 1.4735 | 58.0 |
| Compound C | 0.0055 | 90.7 |
| Compound E | 0.0690 | 81.8 |
| Compound N | 0.052 | 164.8 |

### Example 3. Eyelid Pharmacokinetic of an Exemplary Compound

A formulation of 1% Compound G as a suspension in a suitable vehicle was administered to rabbits as follows. Animals were sedated and the eyelashes and hair on both eyes on both lids trimmed as short as possible. Thirty microliters of the 1% Compound G was drawn up into a pipette and carefully pipetted around the entire eyelid of the animal a few millimeters back from the edge of the lid so as to prevent the material from leaking onto the eye itself. Following application, a cotton swab or eye spear was used to gently try and massage the material into the skin. At the appropriate time points, the animals were euthanized and meibomian glands removed and the concentration of the compound in meibomian glands was measured. The experiment was conducted in triplicate and the results below illustrate mean concentration in nM.

TABLE 3
Mean and standard deviation of Compound
G concentration in rabbit meibomian gland

| Time (h) | Mean (nM) | SD |
|---|---|---|
| 0.5 | 2632.4 | 1952.6 |
| 3 | 3778.7 | 2530.1 |
| 6 | 1054.5 | 761.3 |

As seen in Table above, Compound G demonstrated uptake into the meibomian gland was sustained for at least six hours.

### Example 4. In Vivo Measurement of Lowered Meibum Melting Temperature

For specific compounds Compound B and Compound C, the lowering of rat meibum melting temperature was measured in naive Sprague Dawley rats. The test animals were administered either vehicle or Compound B or Compound C, the meibum collected upon administration of the compounds was analyzed by differential scanning calorimetry to measure the melting point. The lowering of meibum melting point in rats administered Compound B or Compound C were compared to vehicle.

Melting properties of meibum were obtained using differential scanning calorimetry thermograms, recorded on a TA Discovery Q5000 (ThermoAnalytical). Samples were sealed in standard 40 μl aluminum pans and subjected to a heat-cool-heat cycle with melting temperatures being recorded on the second heating ramp. The samples were first heated to 150° C. at 30K/min then cooled −30° C. at 30K/min. Next, the sample was heated to 75° C. with an underlying heating rate of 2K/min, a period of modulation of 60 seconds and a temperature amplitude of modulation of 1° C. Dry N₂ gas, at a flow rate of 50 mL/min was used to purge the DSC equipment during the measurement. Onset of melting and peak temperatures were recorded with peak temperature quoted as the melting point.

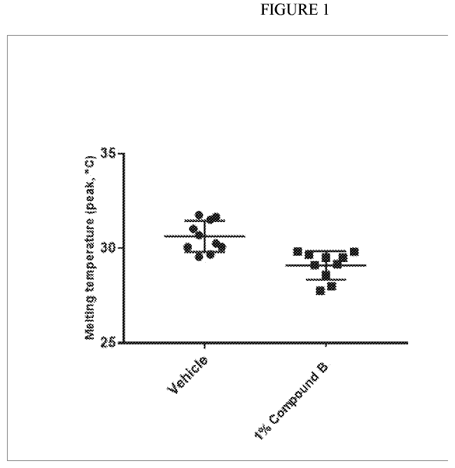

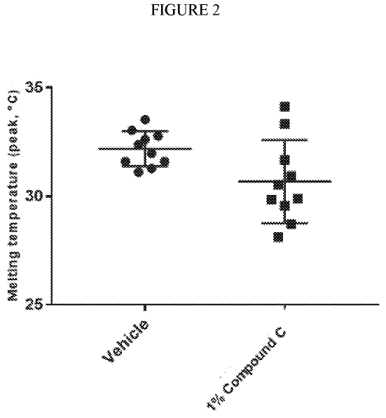

Results from the assay are shown Table 3 and in FIGS. 1 and 2. Results were analyzed using an unpaired t test with Welch's correction.

TABLE 4
Results from in vivo administration of exemplary compounds

| | Compound B | Compound C |
|---|---|---|
| P value | 0.0004 | 0.04 |
| Mean ± SEM of Vehicle | 30.64 ± 0.2612 | 32.19 ± 0.2539 |
| Mean ± SEM of Compound | 29.1 ± 0.2363 | 30.68 ± 0.6065 |
| Change of Tm | −1.534 ± 0.3522 | −1.513 ± 0.6575 |

As seen in the results presented herein, exemplary LXR agonists were able to lower meibum melting temperature in vivo and increase meibum desaturation index in vitro.

### Enumerated Embodiments

In a first aspect, the present invention provides a method of treating meibomian gland dysfunction (MGD) in a subject in need thereof, comprising administering an effective amount of a liver X receptor (LXR) agonist to the subject.

In one embodiment of the first aspect, the LXR agonist is:
2-(4-(3-((2-chloro-3-(trifluoromethyl)benzyl)(2,2-diphenylethyl)amino)propoxy)-1H-indol-1-yl)acetic acid;
2-(tert-butyl)-5-phenyl-4-((4-(piperidin-1-yl)phenyl)amino)isothiazol-3(2H)-one 1,1-dioxide;
(R)-2-(3-(3-((2-chloro-3-(trifluoromethyl)benzyl)(2,2-diphenylethyl)amino)butoxy)phenyl)-2-methylpropanoic acid;
(R)-(2-(4-(4-(hydroxymethyl)-3-(methylsulfonyl)phenyl)-2-(isopropylpiperazin-1-yl)-4-(trifluoromethyl)pyrimidin-5-yl)methanol;
2-(5-(methyl(3-((7-propyl-3-(trifluoromethyl)benzo[d]isoxazol-6-yl)oxy)propyl)amino)pyrazin-2-yl)acetic acid;
N-(4-(1,1,1,3,3,3-hexafluoro-2-hydroxypropan-2-yl)phenyl)-N-(2,2,2-trifluoroethyl)benzenesulfonamide;
ethyl 2-(5-(3′-(methylsulfonyl)-[1,1′-biphenyl]-4-yl)-3-(trifluoromethyl)-1H-pyrazol-1-yl)acetate;
2-(3-(3-((2-chloro-3-(trifluoromethyl)benzyl)(2,2-diphenylethyl)amino)propoxy)phenyl)acetic acid;
2-(2-(2-(2,6-dichlorophenyl)propan-2-yl)-1-(3,3′-difluoro-4′-(hydroxymethyl)-5′-(methylsulfonyl)-[1,1′-biphenyl]-4-yl)-1H-imidazol-4-yl)propan-2-ol;
2-(4-(benzyl(ethyl)amino)-3-chlorophenyl)-1,1,1,3,3,3-hexafluoropropan-2-ol;
2-chloro-4-(5-cyano-6-(4-(2-methyl-2-phenylpropanoyl)piperazin-1-yl)pyridin-3-yl)-N,N-dimethylbenzamide;
2-(2-chloro-4-fluorobenzyl)-3-(4-fluorophenyl)-7-(trifluoromethyl)-2H-indazole;
2-chloro-4-(1′-((2-chlorophenyl)sulfonyl)-[4,4′-bipiperidin]-1-yl)-N,N-dimethylbenzamide;
(R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol;

salts, esters, or co-crystals thereof.

In one embodiment of the first aspect, wherein the LXR agonist is 2-(tert-butyl)-5-phenyl-4-((4-(piperidin-1-yl)phenyl)amino)isothiazol-3(2H)-one 1,1-dioxide; (R)-2-(3-(3-((2-chloro-3-(trifluoromethyl)benzyl)(2,2-diphenylethyl)amino)butoxy)phenyl)-2-methylpropanoic acid; ethyl 2-(5-(3′-(methylsulfonyl)-[1,1′-biphenyl]-4-yl)-3-(trifluoromethyl)-1H-pyrazol-1-yl)acetate; (R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol, or a salt, ester, or co-crystal thereof.

In one embodiment of the first aspect, the method comprises administering about 0.001 mg to about 50 mg of the LXR agonist to the subject.

In one embodiment of the first aspect, the LXR agonist is ocularly administered to the subject.

In one embodiment, the ocular administration is to an eyelid of the subject.

In one embodiment, the ocular administration is to the ocular surface of the subject.

In one embodiment of the first aspect, the LXR agonist is formulated in a pharmaceutically acceptable formulation. In one embodiment, the pharmaceutically acceptable formulation is a solution, suspension, gel, cream, ointment, liposomes, or ocular insert.

In one embodiment, the concentration of the LXR agonist in the pharmaceutically acceptable formulation is about 0.01% w/w to about 10% w/w, or about 0.01% w/w to about 5% w/w, or about 0.05% to about 3% w/w, or about 0.05% w/w to about 0.5% w/w, or about 0.15% w/w, about 0.1% w/w, about 0.5% w/w, about 1.0% w/w about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, or about 6.0% w/w.

In one embodiment of the first aspect, the administration results in an increase in the desaturation index of meibum in the subject.

In one embodiment, the desaturation index of nonpolar lipids generated by human sebaceous gland cell line (SZ95) cells, when measured in vitro is increased by about 10% to about 200%, by about 10% to about 150%, or by about 10% to about 100%.

In one embodiment of the first aspect, the administration results in a decrease in the melting temperature of meibum in the subject. In one embodiment, the melting temperature of meibum is decreased by about 5, about 4, about 3, about 2, or about 1 degrees centigrade.

In one embodiment, the subject is diagnosed with dry eye disease. In one embodiment, the administration decreases the signs and/or symptoms of dry eye disease.

In one embodiment, the administration of the LXR agonist results in one or more of:

increased tear film break-up time of at least about 2, 3, 4, or 5 seconds;

meibomian gland expression grading improvement by 1 or 2 or 3 grades;

increased tear meniscus of at least about 10%;

increased tear film break-up time of at least about 2, 3, 4, or 5 seconds;

decreased corneal fluorescein staining of at least about 10%, or

increased Schirmer test score of at least about 2 mm.

In one embodiment, the administration results in reduced incidence of at least about 10% in one or more of ocular dryness, ocular discomfort or pain, eye itchiness, blurry vision, heavy or fatigued eyes, watery eyes, ocular hyperemia, ocular burning or stinging, grittiness or foreign body sensation, or photophobia or light sensitivity, crusty or red or swollen eyelids or eyelid margins, sensitivity to environmental factors such as wind or low humidity, loss of tolerability to contact lens use.

In one embodiment of the first aspect, the method further comprises administering an additional therapeutic agent to the subject. In one embodiment, the additional therapeutic agent is a retinoid X receptor (RXR) agonist, an ophthalmic steroid, a keratolytic agent, a dry eye agent, or a tetracycline antibiotic. In a particular embodiment, the RXR agonist is vitamin A, retinoic acid, phytanic acid, lithocholic acid, bexarotene, docosahexaenoic acid, flurobexarotene, or pharmaceutically acceptable salts thereof;

the ophthalmic steroid is dexamethasone, fluocinolone, loteprednol, difluprednate, fluorometholone, prednisolone, prednisone, medrysone, triamcinolone, betamethasone, rimexolone, or pharmaceutically acceptable salts thereof;
the dry eye agent is lifitegrast or cyclosporine; or
the keratolytic agent is selenium disulfide, salicylic acid, glycolic acid, or pharmaceutically acceptable salts thereof.

In a second aspect, the present invention provides a method of upregulating stearoyl-CoA desaturase 1 (SCD1) in a subject suffering from meibomian gland dysfunction (MGD), comprising administering an liver X receptor (LXR) agonist to the subject.

In one embodiment of the second aspect, the LXR agonist is:
2-(4-(3-((2-chloro-3-(trifluoromethyl)benzyl)(2,2-diphenylethyl)amino)propoxy)-1H-indol-1-yl)acetic acid;
2-(tert-butyl)-5-phenyl-4-((4-(piperidin-1-yl)phenyl)amino)isothiazol-3(2H)-one 1,1-dioxide;
(R)-2-(3-(3-((2-chloro-3-(trifluoromethyl)benzyl)(2,2-diphenylethyl)amino)butoxy)phenyl)-2-methylpropanoic acid;
(R)-(2-(4-(4-(hydroxymethyl)-3-(methylsulfonyl)phenyl)-2-(isopropylpiperazin-1-yl)-4-(trifluoromethyl)pyrimidin-5-yl)methanol;
2-(5-(methyl(3-((7-propyl-3-(trifluoromethyl)benzo[d]isoxazol-6-yl)oxy)propyl)amino)pyrazin-2-yl)acetic acid;
N-(4-(1,1,1,3,3,3-hexafluoro-2-hydroxypropan-2-yl)phenyl)-N-(2,2,2-trifluoroethyl)benzenesulfonamide;
ethyl 2-(5-(3′-(methylsulfonyl)-[1,1′-biphenyl]-4-yl)-3-(trifluoromethyl)-1H-pyrazol-1-yl)acetate;
2-(3-(3-((2-chloro-3-(trifluoromethyl)benzyl)(2,2-diphenylethyl)amino)propoxy)phenyl)acetic acid;
2-(2-(2-(2,6-dichlorophenyl)propan-2-yl)-1-(3,3′-difluoro-4′-(hydroxymethyl)-5′-(methylsulfonyl)-[1,1′-biphenyl]-4-yl)-1H-imidazol-4-yl)propan-2-ol;
2-(4-(benzyl(ethyl)amino)-3-chlorophenyl)-1,1,1,3,3,3-hexafluoropropan-2-ol; 2-chloro-4-(5-cyano-6-(4-(2-methyl-2-phenylpropanoyl)piperazin-1-yl)pyridin-3-yl)-N,N-dimethylbenzamide;
2-(2-chloro-4-fluorobenzyl)-3-(4-fluorophenyl)-7-(trifluoromethyl)-2H-indazole; 2-chloro-4-(1′-((2-chlorophenyl)sulfonyl)-[4,4′-bipiperidin]-1-yl)-N,N-dimethylbenzamide;
(R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol,

salts, esters, or co-crystals thereof.

In one embodiment of the second aspect, the LXR agonist is 2-(tert-butyl)-5-phenyl-4-((4-(piperidin-1-yl)phenyl)amino)isothiazol-3(2H)-one 1,1-dioxide; (R)-2-(3-(3-((2-chloro-3-(trifluoromethyl)benzyl)(2,2-diphenylethyl)amino)butoxy)phenyl)-2-methylpropanoic acid; ethyl 2-(5-(3′-(methylsulfonyl)-[1,1′-biphenyl]-4-yl)-3-(trifluoromethyl)-1H-pyrazol-1-yl)acetate; (R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol, or a salt, ester, or co-crystal thereof.

In one embodiment of the second aspect, the method comprises administering about 0.001 mg to about 50 mg of the LXR agonist to the subject.

In one embodiment of the second aspect, the LXR agonist is ocularly administered to the subject. In one embodiment the ocular administration is to an eyelid of the subject. In one embodiment, the ocular administration is to the ocular surface of the subject.

In one embodiment of the second aspect, the LXR agonist is formulated in a pharmaceutically acceptable formulation.

In one embodiment of the second aspect, the pharmaceutically acceptable formulation is a solution, suspension, gel, cream, ointment, liposomes, or ocular insert. In one embodiment, the concentration of the LXR agonist in the pharmaceutically acceptable formulation is about 0.01% w/w to about 10% w/w, or about 0.01% w/w to about 5% w/w, or about 0.05% to about 3% w/w, or about 0.05% w/w to about 0.5% w/w, or about 0.15% w/w, about 0.1% w/w, about 0.5% w/w, about 1.0% w/w about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, or about 6.0% w/w.

In one embodiment of the second aspect, the administration results in an increase in the desaturation index of meibum in the subject. In one embodiment, the desaturation index of nonpolar lipids generated by human sebaceous gland cell line (SZ95) cells when measured in vitro is increased by about 10% to about 200%, by about 10% to about 150%, or by about 10% to about 100%.

In one embodiment of the second aspect, the administration results in a decrease in the melting temperature of meibum in the subject.

In one embodiment of the second aspect, the melting temperature of meibum is decreased by about 5, about 4, about 3, about 2, or about 1 degrees centigrade.

In one embodiment of the second aspect, the subject is diagnosed with dry eye disease. In one embodiment, the administration decreases the signs and/or symptoms of dry eye disease.

In one embodiment of the second aspect, the administration results in one or more of:

increased tear film break-up time of at least about 2, 3, 4, or 5 seconds;

meibomian gland expression grading improvement by 1 or 2 or 3 grades;

increased tear meniscus of at least about 10%;

increased tear film break-up time of at least about 2, 3, 4, or 5 seconds;

decreased corneal fluorescein staining of at least about 10%, or

increased Schirmer test score of at least about 2 mm.

In one embodiment of the second aspect, the administration results in reduced incidence of at least about 10% in one or more of ocular dryness, ocular discomfort or pain, eye itchiness, blurry vision, heavy or fatigued eyes, watery eyes, ocular hyperemia, ocular burning or stinging, grittiness or foreign body sensation, or photophobia or light sensitivity, crusty or red or swollen eyelids or eyelid margins, sensitivity to environmental factors such as wind or low humidity, loss of tolerability to contact lens use.

In one embodiment of the second aspect, the method comprises administering an additional therapeutic agent to the subject. In one embodiment, the additional therapeutic agent is a retinoid X receptor (RXR) agonist, an ophthalmic steroid, a keratolytic agent, a dry eye agent, or a tetracycline antibiotic. In one embodiment, the RXR agonist is vitamin A, retinoic acid, phytanic acid, lithocholic acid, bexarotene, docosahexaenoic acid, flurobexarotene, or pharmaceutically acceptable salts thereof;

the ophthalmic steroid is dexamethasone, fluocinolone, loteprednol, difluprednate, fluorometholone, prednisolone, prednisone, medrysone, triamcinolone, betamethasone, rimexolone, or pharmaceutically acceptable salts thereof;
the dry eye agent is lifitegrast or cyclosporine; or
the keratolytic agent is selenium disulfide, salicylic acid, glycolic acid, or pharmaceutically acceptable salts thereof.

In a third aspect, the present invention provides a method of reducing the symptoms of meibomian gland dysfunction (MGD) in a subject in need thereof, comprising administering an effective amount of a liver X receptor (LXR) agonist to the subject.

In one embodiment of the third aspect, the LXR agonist is:
2-(4-(3-((2-chloro-3-(trifluoromethyl)benzyl)(2,2-diphenylethyl)amino)propoxy)-1H-indol-1-yl)acetic acid;
2-(tert-butyl)-5-phenyl-4-((4-(piperidin-1-yl)phenyl)amino)isothiazol-3(2H)-one 1,1-dioxide;
(R)-2-(3-(3-((2-chloro-3-(trifluoromethyl)benzyl)(2,2-diphenylethyl)amino)butoxy)phenyl)-2-methylpropanoic acid;
(R)-(2-(4-(4-(hydroxymethyl)-3-(methylsulfonyl)phenyl)-2-isopropylpiperazin-1-yl)-4-(trifluoromethyl)pyrimidin-5-yl)methanol;
2-(5-(methyl(3-((7-propyl-3-(trifluoromethyl)benzo[d]isoxazol-6-yl)oxy)propyl)amino)pyrazin-2-yl)acetic acid;
N-(4-(1,1,1,3,3,3-hexafluoro-2-hydroxypropan-2-yl)phenyl)-N-(2,2,2-trifluoroethyl)benzenesulfonamide;
ethyl 2-(5-(3′-(methylsulfonyl)-[1,1′-biphenyl]-4-yl)-3-(trifluoromethyl)-1H-pyrazol-1-yl)acetate;
2-(3-(3-((2-chloro-3-(trifluoromethyl)benzyl)(2,2-diphenylethyl)amino)propoxy)phenyl)acetic acid;
2-(2-(2-(2,6-dichlorophenyl)propan-2-yl)-1-(3,3′-difluoro-4′-(hydroxymethyl)-5′-(methylsulfonyl)-[1,1′-biphenyl]-4-yl)-1H-imidazol-4-yl)propan-2-ol;
2-(4-(benzyl(ethyl)amino)-3-chlorophenyl)-1,1,1,3,3,3-hexafluoropropan-2-ol;
2-chloro-4-(5-cyano-6-(4-(2-methyl-2-phenylpropanoyl)piperazin-1-yl)pyridin-3-yl)-N,N-dimethylbenzamide;
2-(2-chloro-4-fluorobenzyl)-3-(4-fluorophenyl)-7-(trifluoromethyl)-2H-indazole;
2-chloro-4-(1′-((2-chlorophenyl)sulfonyl)-[4,4′-bipiperidin]-1-yl)-N,N-dimethylbenzamide;
(R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol,

salts, esters, or co-crystals thereof.

In one embodiment of the third aspect, the LXR agonist is 2-(tert-butyl)-5-phenyl-4-((4-(piperidin-1-yl)phenyl)amino)isothiazol-3(2H)-one 1,1-dioxide; (R)-2-(3-(3-((2-chloro-3-(trifluoromethyl)benzyl)(2,2-diphenylethyl)amino)butoxy)phenyl)-2-methylpropanoic acid; ethyl 2-(5-(3′-(methylsulfonyl)-[1,1′-biphenyl]-4-yl)-3-(trifluoromethyl)-1H-pyrazol-1-yl)acetate; (R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol, or a salt, ester, or co-crystal thereof.

In one embodiment of the third aspect, the method comprises administering about 0.001 mg to about 50 mg of the LXR agonist to the subject.

In one embodiment of the third aspect, the LXR agonist is ocularly administered to the subject. In one embodiment, the ocular administration is to an eyelid of the subject.

In one embodiment, the ocular administration is to the ocular surface of the subject.

All publications and patent documents cited herein are incorporated herein by reference as if each such publication or document was specifically and individually indicated to be incorporated herein by reference. The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the invention that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein. The claims should not be read as limited to the described order or elements unless stated to that effect. It should be understood that various changes in form and detail may be made without departing from the scope of the appended claims.

What is claimed is:

1. A method of treating meibomian gland dysfunction (MGD) in a subject in need thereof, comprising administering an effective amount of a liver X receptor (LXR) agonist to the subject.

2. The method according to claim 1, wherein the LXR agonist is:
2-(4-(3-((2-chloro-3-(trifluoromethyl)benzyl)(2,2-diphenylethyl)amino)propoxy)-1H-indol-1-yl)acetic acid;
2-(tert-butyl)-5-phenyl-4-((4-(piperidin-1-yl)phenyl)amino)isothiazol-3(2H)-one 1,1-dioxide;
(R)-2-(3-(3-((2-chloro-3-(trifluoromethyl)benzyl)(2,2-diphenylethyl)amino)butoxy)phenyl)-2-methylpropanoic acid;
(R)-(2-(4-(4-(hydroxymethyl)-3-(methylsulfonyl)phenyl)-2-isopropylpiperazin-1-yl)-4-(trifluoromethyl)pyrimidin-5-yl)methanol;
2-(5-(methyl(3-((7-propyl-3-(trifluoromethyl)benzo[d]isoxazol-6-yl)oxy)propyl)amino)pyrazin-2-yl)acetic acid;
N-(4-(1,1,1,3,3,3-hexafluoro-2-hydroxypropan-2-yl)phenyl)-N-(2,2,2-trifluoroethyl)benzenesulfonamide;
ethyl 2-(5-(3′-(methylsulfonyl)-[1,1′-biphenyl]-4-yl)-3-(trifluoromethyl)-1H-pyrazol-1-yl)acetate;
2-(3-(3-((2-chloro-3-(trifluoromethyl)benzyl)(2,2-diphenylethyl)amino)propoxy)phenyl)acetic acid;
2-(2-(2-(2,6-dichlorophenyl)propan-2-yl)-1-(3,3′-difluoro-4′-(hydroxymethyl)-5′-(methylsulfonyl)-[1,1′-biphenyl]-4-yl)-1H-imidazol-4-yl)propan-2-ol;
2-(4-(benzyl(ethyl)amino)-3-chlorophenyl)-1,1,1,3,3,3-hexafluoropropan-2-ol;
2-chloro-4-(5-cyano-6-(4-(2-methyl-2-phenylpropanoyl)piperazin-1-yl)pyridin-3-yl)-N,N-dimethylbenzamide;
2-(2-chloro-4-fluorobenzyl)-3-(4-fluorophenyl)-7-(trifluoromethyl)-2H-indazole;
2-chloro-4-(1′-((2-chlorophenyl)sulfonyl)-[4,4′-bipiperidin]-1-yl)-N,N-dimethylbenzamide;
(R)-2-(2-(8-(hydroxymethyl)-1-isopropyl-7-(methylsulfonyl)-3,4-dihydrobenzo[4,5]imidazo[1,2-a]pyrazin-2(1H)-yl)-4-(trifluoromethyl)pyrimidin-5-yl)propan-2-ol;
salts, esters, or co-crystals thereof.

3. The method according any of the preceding claims, comprising administering about 0.001 mg to about 50 mg of the LXR agonist to the subject.

4. The method of any of the preceding claims, wherein the LXR agonist is ocularly administered to the subject.

5. The method of any of the preceding claims, wherein the LXR agonist is formulated in a pharmaceutically acceptable formulation.

6. The method according to claim 5, wherein the concentration of the LXR agonist in the pharmaceutically acceptable formulation is about 0.01% w/w to about 10% w/w, or about 0.01% w/w to about 5% w/w, or about 0.05% to about 3% w/w, or about 0.05% w/w to about 0.5% w/w, or about 0.15% w/w, about 0.1% w/w, about 0.5% w/w, about 1.0% w/w about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, or about 6.0% w/w.

7. The method according to any of the preceding claims, wherein the administration results in an increase in the desaturation index of meibum in the subject.

8. The method according to any of the preceding claims, wherein the administration results in a decrease in the melting temperature of meibum in the subject.

9. The method according to any of the preceding claims, wherein the subject is diagnosed with dry eye disease.

10. The method according to claim 9, wherein the administration decreases the signs and/or symptoms of dry eye disease.

11. The method according to any of the preceding claims, further comprising administering an additional therapeutic agent to the subject.

12. A method of upregulating stearoyl-CoA desaturase 1 (SCD1) in a subject suffering from meibomian gland dysfunction (MGD), comprising administering an liver X receptor (LXR) agonist to the subject.

13. The method according to claim 12, comprising administering about 0.001 mg to about 50 mg of the LXR agonist to the subject.

14. The method of any of claim 12 or 13, wherein the LXR agonist is ocularly administered to the subject.

15. The method according to any of claims 12-14, wherein the administration results in an increase in the desaturation index of meibum in the subject.

16. The method according to any of claims 12-15, wherein the administration results in a decrease in the melting temperature of meibum in the subject.

17. The method according to any of claims 12-16, wherein the subject is diagnosed with dry eye disease.

18. The method according to any of claims 12-17, further comprising administering an additional therapeutic agent to the subject.

19. A method of reducing the symptoms of meibomian gland dysfunction (MGD) in a subject in need thereof, comprising administering an effective amount of a liver X receptor (LXR) agonist to the subject.

20. The method of claim 19, wherein the LXR agonist is ocularly administered to the subject.
